(19) United States
(12) Patent Application Publication
(10) Pub. No.: US 2019/0094179 A1
(43) Pub. Date: Mar. 28, 2019

(54) METHOD FOR SIMPLE FLUIDIC ADDRESSING OF A NANOPORE

(71) Applicant: Applied Materials, Inc., Santa Clara, CA (US)

(72) Inventors: Mangesh BANGAR, San Jose, CA (US); Joseph R. JOHNSON, Redwood City, CA (US)

(21) Appl. No.: 16/041,429

(22) Filed: Jul. 20, 2018

Related U.S. Application Data
(60) Provisional application No. 62/561,970, filed on Sep. 22, 2017.

(51) Int. Cl. G01N 27/447; C12Q 1/6869; G01N 33/487

(52) U.S. Cl. CPC G01N 27/44791; B82Y 15/00; G01N 33/48721; C12Q 1/6869

(57) ABSTRACT

Aspects disclosed herein relate to methods of high-volume manufacturing of an array of biological sensing devices on a substrate, each of the biological sensing devices having a vertical or horizontal membrane having one or more solid-state nanopores therethrough, and methods for simple fluidic addressing of each nanopore. In one aspect, a method for forming a nanopore by applying a voltage from a positive electrode to a negative electrode through a free-standing membrane is disclosed. In other aspects, methods for forming a plurality of nanopores on a wafer are disclosed. In another aspect, a single-sided processing method for forming a nanopore device is disclosed to provide a device having baths on either side of a nanopore, which are addressable from a single side of the substrate. In yet another aspect, a method for fluidically addressing a plurality of nanopore devices is disclosed.

### CROSS-REFERENCE TO RELATED APPLICATIONS

This application claims benefit of U.S. Provisional Patent Application Ser. No. 62/561,970, filed on Sep. 22, 2017, which is herein incorporated by reference in its entirety.

### BACKGROUND

### Field

Aspects disclosed herein relate to methods of high-volume manufacturing of an array of biological sensing devices on a substrate, each of the biological sensing devices having a vertical or horizontal membrane having one or more solid-state nanopores therethrough, and methods for simple fluidic addressing of each nanopore.

### Description of the Related Art

Nanopores are widely used for applications such as deoxyribonucleic acid (DNA) and ribonucleic acid (RNA) sequencing. In one example, nanopore sequencing is performed using an electrical detection method, which generally includes transporting an unknown sample through the nanopore, which is immersed in a conducting fluid, and applying electric potential across the nanopore. Electric current resulting from the conduction of ions through the nanopore is measured. The magnitude of the electric current density across a nanopore surface depends on the nanopore dimensions and the composition of the sample, such as DNA or RNA, which is occupying the nanopore at the time. Different nucleotides cause characteristic changes in electric current density across nanopore surfaces. These electric current changes are measured and used to sequence the DNA or RNA sample.

Various methods have been used for biological sequencing. Sequencing by synthesis, or second generation sequencing, is used to identify which bases have attached to a single strand of DNA. Third generation sequencing, which generally includes threading an entire DNA strand through a single pore, is used to directly read the DNA. Some sequencing methods require the DNA or RNA sample to be cut up and then reassembled. Additionally, some sequencing methods use biological membranes and biological pores, which have shelf lives and must be kept cold prior to use.

Solid-state nanopores, which are nanometer-sized pores formed on a free-standing membrane such as silicon nitride or silicon oxide, have recently been used for sequencing. Current solid-state nanopore fabrication methods, such as using a tunneling electron microscope, focused ion beam, or electron beam, however, cannot easily and cheaply achieve the size and position control requirements necessary for manufacturing arrays of nanopores. Additionally, current nanopore fabrication methods are time consuming. Moreover, current free-standing membrane fabrication methods are manual, time consuming and costly, and cannot be efficiently used to repetitively form a free-standing membrane, such as a vertical membrane, with the optimum thinness for DNA or RNA sequencing.

Therefore, there is a need in the art for methods of high scale manufacturing vertical or horizontal membranes having one or more solid-state nanopores therethrough, and methods for fluidic addressing of the nanopores.

### SUMMARY

Aspects disclosed herein relate to methods of high-volume manufacturing of an array of biological sensing devices on a substrate, each of the biological sensing devices having a vertical or horizontal membrane having one or more solid-state nanopores therethrough, and methods for simple fluidic addressing of each nanopore. In one aspect, a method for forming a nanopore by applying a voltage from a positive electrode to a negative electrode through a free-standing membrane is disclosed. In other aspects, methods for forming a plurality of nanopores on a wafer are disclosed. In another aspect, a single-sided processing method for forming a nanopore device is disclosed to provide a device having a bath on either side of a nanopore, which are addressable from a single side of the substrate. In yet another aspect, a method for fluidically addressing a plurality of nanopore devices is disclosed.

In one aspect, a method for forming a biological sequencing device is disclosed. The method includes forming a plurality of nanopore devices on a substrate, each nanopore device having a first bath and a second bath, forming a first bath reservoir in fluid communication with each of the first baths through a plurality of first channels, forming a second bath reservoir in fluid communication with each of the second baths through a plurality of second channels.

In another aspect, a method for forming a nanopore device is disclosed. The method includes depositing a first selectively-etchable material over a first non-selectively etchable material on a substrate, depositing a dielectric material over the first selectively-etchable material, depositing a second selectively-etchable material over the dielectric material, depositing a second non-selectively etchable material over the second selectively-etchable material, and selectively etching the first selectively-etchable material and the second selectively-etchable material to form a first bath and a second bath on a single side of the substrate and on either side of the dielectric material.

In yet another aspect, a device for biological sequencing applications is disclosed. The device includes a plurality of nanopore devices, a first bath reservoir, and a second bath reservoir. The first bath reservoir being fluidically coupled to each of the plurality of nanopore devices through a series of first channels and the second bath reservoir being fluidically coupled to each of the plurality of nanopore devices through a series of second channels.

### BRIEF DESCRIPTION OF THE DRAWINGS

So that the manner in which the above recited features of the present disclosure can be understood in detail, a more particular description of the disclosure, briefly summarized above, may be had by reference to aspects, some of which are illustrated in the appended drawings. It is to be noted, however, that the appended drawings illustrate only exemplary aspects and are therefore not to be considered limiting of its scope, and may admit to other equally effective aspects.

FIGS. 1A-1D depict cross-sectional views of a substrate at various stages of a method disclosed herein.

FIG. 2 is a top-down view of a wafer having a plurality of nanopore devices thereon.

FIG. 3 is a cross-sectional view of a portion of the wafer of FIG. 2 having two nanopore devices thereon during a DNA sequencing process.

FIGS. 4A-4C depict top down views of various configurations of a portion of the wafer of FIG. 2.

FIGS. 5A-5M depict cross-sectional views cross-sectional views of a substrate for biological sequencing applications at various stages of a method disclosed herein.

FIG. 6A is a top down view of a plurality of substrates connected to a first bath reservoir and a second bath reservoir by a plurality of channels.

FIG. 6B is a cross-sectional view of one of the substrates connected to the first bath reservoir and the second bath reservoir.

FIG. 7 is a three-dimensional view of a substrate for biological sequencing applications.

To facilitate understanding, identical reference numerals have been used, where possible, to designate identical elements that are common to the figures. It is contemplated that elements and features of one aspect may be beneficially incorporated in other aspects without further recitation.

### DETAILED DESCRIPTION

Aspects disclosed herein relate to methods of high-volume manufacturing of an array of biological sensing devices on a substrate, each of the biological sensing devices having a vertical or horizontal membrane having one or more solid-state nanopores therethrough, and methods for simple fluidic addressing of each nanopore. In one aspect, a method for forming a nanopore by applying a voltage from a positive electrode to a negative electrode through a free-standing membrane is disclosed. In other aspects, methods for forming a plurality of nanopores on a wafer are disclosed. In another aspect, a single-sided processing method for forming a nanopore device is disclosed to provide a device having baths on either side of a nanopore, which are addressable from a single side of the substrate. In yet another aspect, a method for fluidically addressing a plurality of nanopore devices is disclosed.

Methods described herein refer to formation of solid-state nanopores on a semiconductor substrate as an example. It is also contemplated that the described methods are useful to form other pore-like structures on various materials, including solid state and biological materials. Methods described herein also refer to formation of trenches as an example; however, other etched features and any combinations thereof are also contemplated. For illustrative purposes, a silicon substrate with a silicon oxide dielectric layer is described; however, any suitable substrate materials and dielectric materials are also contemplated. Additionally, methods described herein refer to a topside and a backside of the substrate. The topside and backside generally refer to opposite sides of the substrate and do not necessarily require an upward or downward orientation.

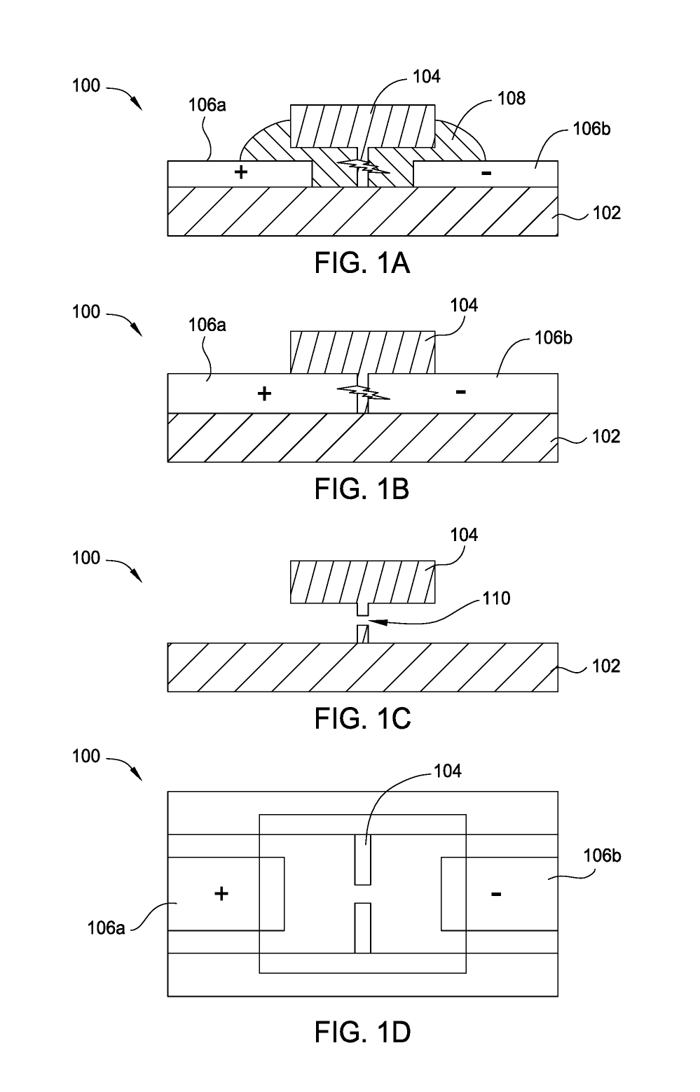

FIGS. 1A-1D depict cross-sectional views of a substrate 100 on which one or more nanopores are formed at various stages of a method disclosed herein.

The substrate 100 generally includes a silicon layer 102. A free-standing membrane 104 is deposited on the substrate 100. FIGS. 1A-1D show a vertical free-standing membrane for illustrative purposes. However, horizontal free-standing membranes are also contemplated herein. The free-standing membrane 104 is generally deposited or formed by any suitable method, examples of which are disclosed below.

In one aspect, the method begins by depositing a positive electrode 106a and a negative electrode 106b on either side of the free-standing membrane 104. As shown in FIG. 1A, the positive electrode 106a and the negative electrode 106b are deposited a distance away from the free-standing membrane 104. A conductive fluid 108 is deposited within the space between each of the electrodes 106a, 106b and the free-standing membrane 104. As shown in FIG. 1B, the positive electrode 106a and the negative electrode 106b are deposited adjacent to the free-standing membrane 104. A voltage is applied from the positive electrode 106a to the negative electrode 106b to breakdown the free-standing membrane 104 and form a nanopore 110 formed therethrough, as shown in FIG. 1C and FIG. 1D, which is a top-down view of the substrate 100 having the nanopore 110 therethrough. Once the nanopore 110 is formed through the free-standing membrane 104 on the substrate 100, the substrate 100 may be used as a device for sequencing applications, such as biological sequencing, for example for DNA or RNA sequencing. For example, continuous or intermittent current sensing is generally performed to determine a size of the DNA or RNA sample in the nanopore 110.

The positive electrode 106a and the negative electrode 106b are optionally selectively removed, as shown in FIG. 1C. In one aspect, the conductive fluid 108 is deposited by inkjet printing. In one aspect, the voltage is applied continuously. In another aspect, the voltage is pulsed. The voltage is generally any voltage greater than or equal to the breakdown voltage of the material of the free-standing membrane 104. The size, or diameter, of the nanopore 110 generally increases as the voltage increases above the breakdown voltage of the material and as the time the voltage is applied is increased.

The size and position of the nanopore 110 are well controlled. A well-controlled size of the nanopore 110 is generally a diameter suitable for sequencing a sample of a certain size. In one aspect, the size of the nanopore 110 is about 100 nanometers (nm) or less. In one aspect, the size of the nanopore 110 is between about 0.5 nm and about 5 nm, for example between about 1 nm and about 3 nm, such as 2 nm. In another aspect, the size of the nanopore 110 is between about 1.5 nm and about 1.8 nm, such as about 1.6 nm, which is roughly the size single stranded DNA. In another aspect, the size of the nanopore 110 is between about 2 nm and about 3 nm, such as about 2.8 nm, which is roughly the size of double-stranded DNA. A well-controlled position of the nanopore 110 is generally any position on the substrate which is suitable for configuration of one or more nanopores.

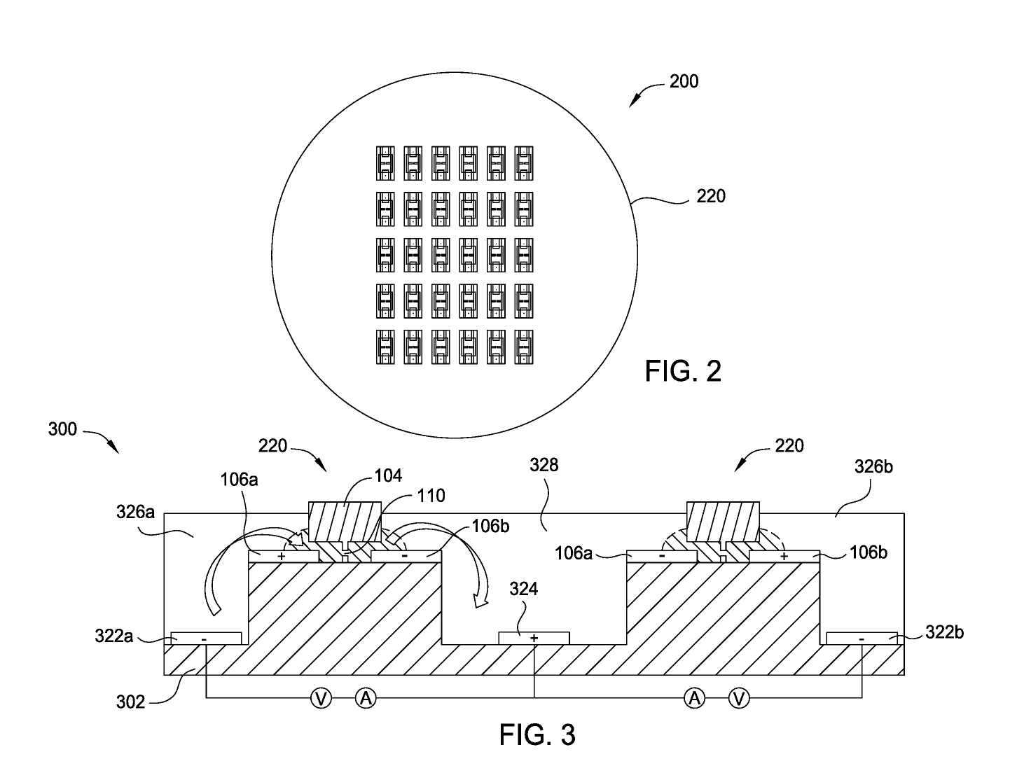

FIG. 2 is a top-down view of a wafer 200 having a plurality of nanopore devices 220 thereon. Each nanopore device 220 has at least one nanopore 110. In one aspect, each nanopore device 220 has a single nanopore 110. In another aspect, each nanopore device 220 has multiple nanopores 110. In one aspect, the nanopore devices 220 are the substrates 100 described above, the manufacturing method of which has been multiplied across the wafer 200 to bring high volume manufacturing to nanopore fabrication. In another aspect, the nanopore devices 220 are similar devices capable of biological sequencing, formed according to any suitable nanopore manufacturing methods. The wafer 200 is generally formed using wafer fabrication equipment and may include as many as hundreds to thousands to millions of densely-packed nanopore devices 220. The nanopore devices 220 can be diced up and sold individually, grouped on the wafer 200 in an arrangement so they can be diced in groups and then inserted into a DNA sequencing device, or be left on the wafer 200 where the whole wafer 200 is the DNA sequencing device. The array of nanopore devices 220 on the wafer 200 may be used to parallelize sequencing, making sequencing times faster, or may be used to perform multiple tests (including other biological tests) on a single wafer 200.

After the nanopore devices 220 have been deposited or formed on the wafer 200, a sample-containing solution is generally deposited over one side of the nanopore 110 and a sample-free solution is deposited over the other side of the nanopore 110. In the example of DNA sequencing, a DNA-containing solution is deposited over one side of the nanopore 110 and a DNA-free solution deposited over the other side of the nanopore 110. In one aspect, the deposited solutions are added separately for each nanopore 110. In another aspect, a common DNA-containing solution is added to all of the negative electrode (anode) sides and a common pool of DNA-free solution is added to all of the positive electrode (cathode) sides, or vice versa. In one aspect, receptacles for the DNA solution are fabricated into the wafer 200. In another aspect, receptacles for the DNA solution are fabricated from a different interface, such as a DNA synthesis plate.

FIG. 3 is a cross-sectional view of a portion 300 of the wafer 200 having two nanopore devices 220 thereon during a DNA sequencing process. As shown in FIG. 3, the two nanopore devices 220 each have a cathode 322a, 322b, respectively, and share a common anode 324. A DNA-containing solution, which is generally DNA in a conductive liquid, is added to the cathode reservoirs 326a, 326b. During sequencing, a voltage is applied across the nanopore and the DNA flows from the cathode reservoirs 326a, 326b to the anode reservoir 328 through the nanopores 110. When the DNA flows through the nanopore 110, the electrical current through the nanopore 110 is measured such that the DNA sample can be sequenced. Since the nanopore devices 220 are connected, the DNA sequencing process is generally parallelized.

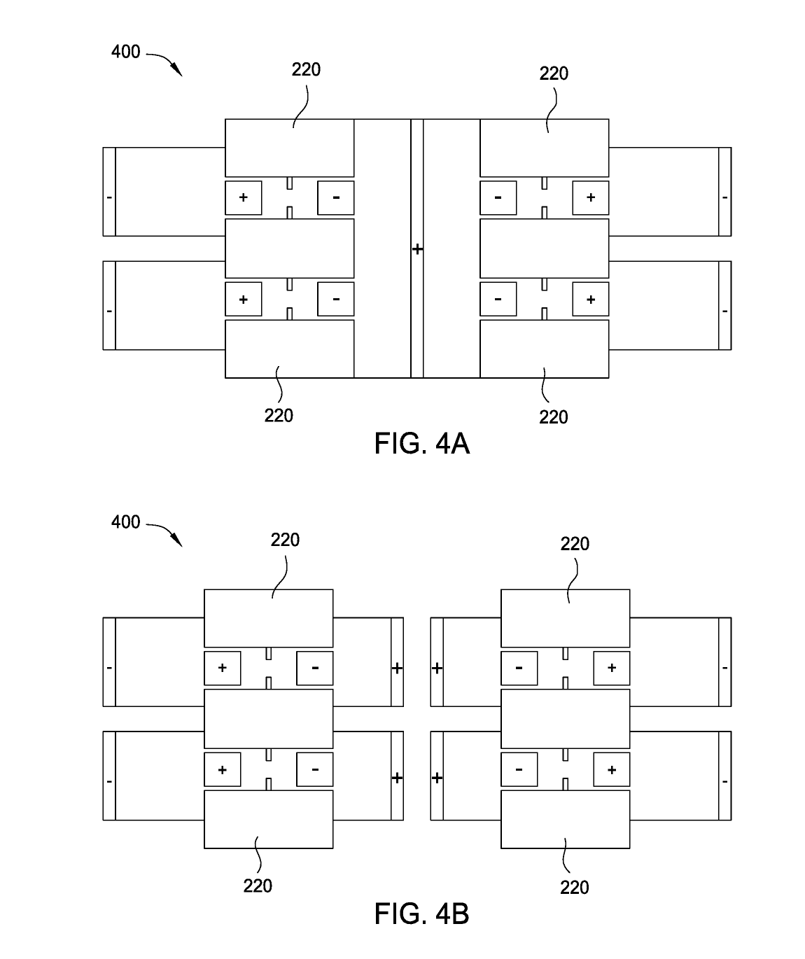

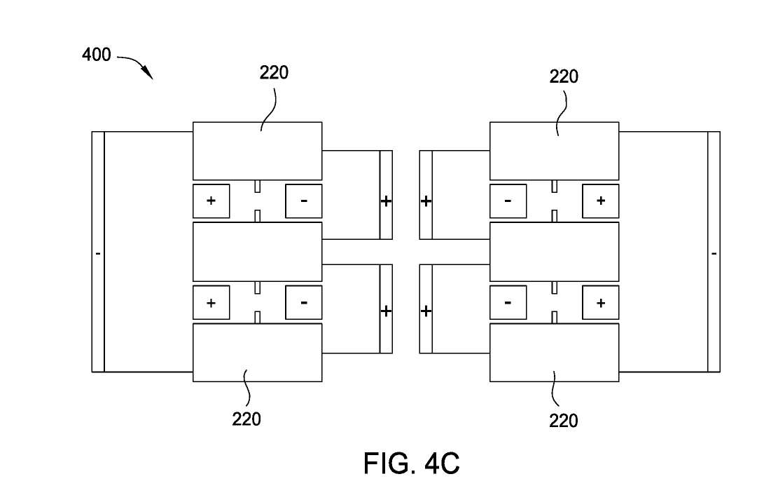

FIGS. 4A-4C depict top down views of various configurations of a portion 400 of the wafer 200. As shown in FIG. 4A, the nanopore devices 220 share a common anode reservoir or positive voltage. As shown in FIG. 4B, each of the nanopore devices 220 has its own cathode reservoir and its own anode reservoir. As shown in FIG. 4C, a first two of the nanopore devices 220 share an anode reservoir and a second two of the nanopore devices 220 share another anode reservoir, and each of the nanopore devices 220 has its own cathode reservoir. The example of FIG. 4A is generally useful for a single DNA sequence, which is being verified. The example of FIG. 4B is generally useful for selecting individual pools of sequenced DNA. The example of FIG. 4C is generally useful for selecting high quality sequenced DNA from a large pool of similar DNA.

In some aspects, such as those shown in FIGS. 4A-4C, each nanopore is individually electrically addressable. In order to be electrically addressable, the nanopore needs at least its own reservoir, such as a cathode or an anode reservoir, on one side of the nanopore. This individual electrical addressability is useful for adjusting the speed of sequencing through the nanopore, as well as preventing the mixing of signals.

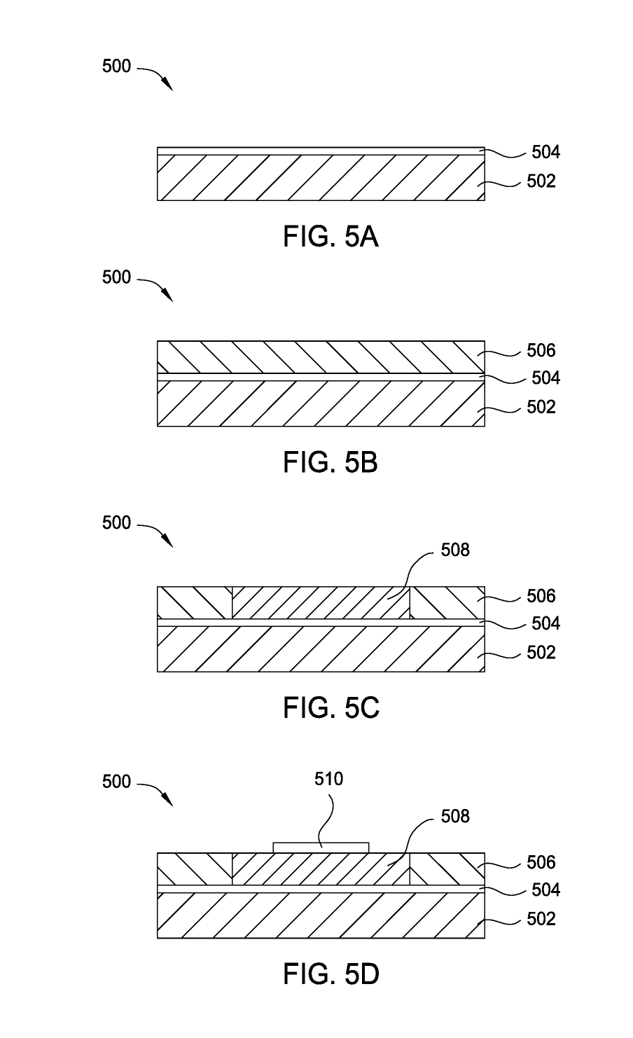

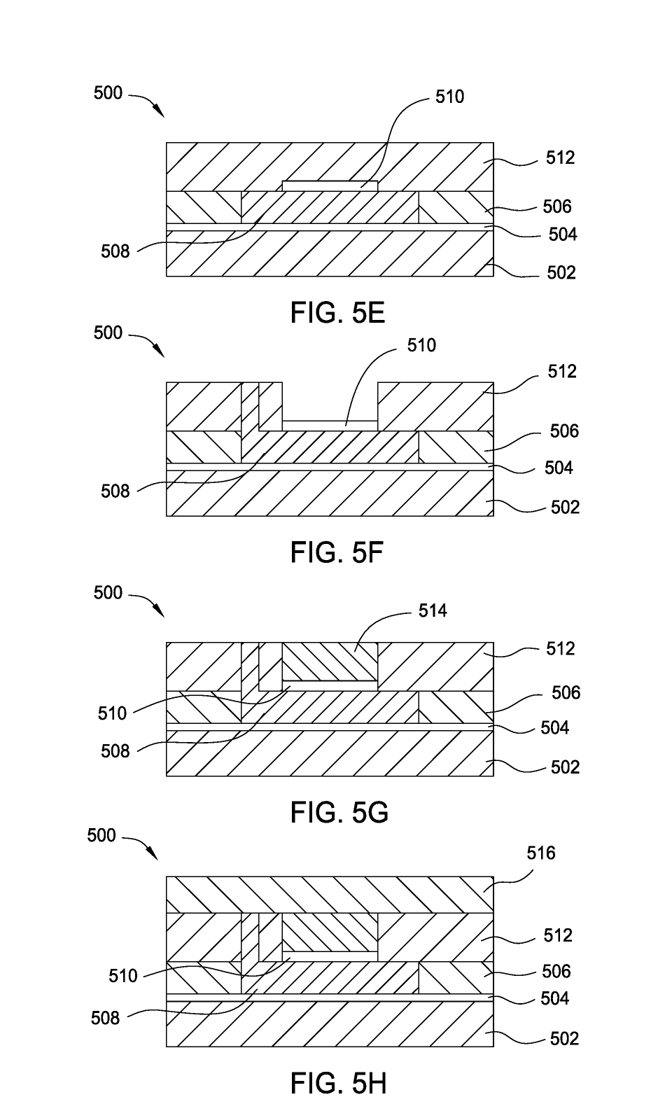

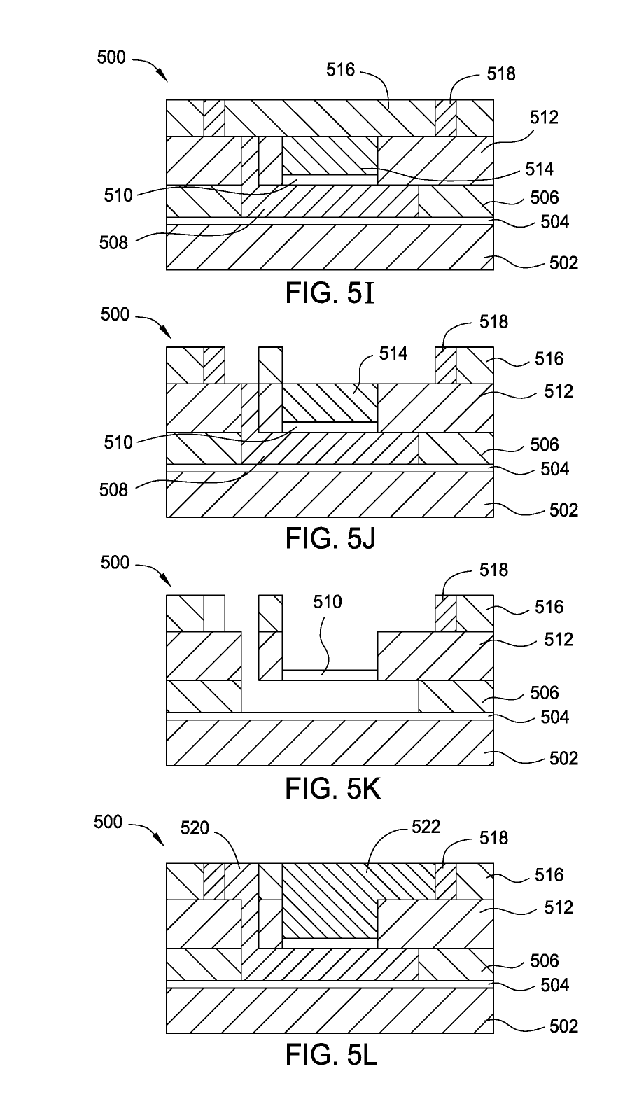

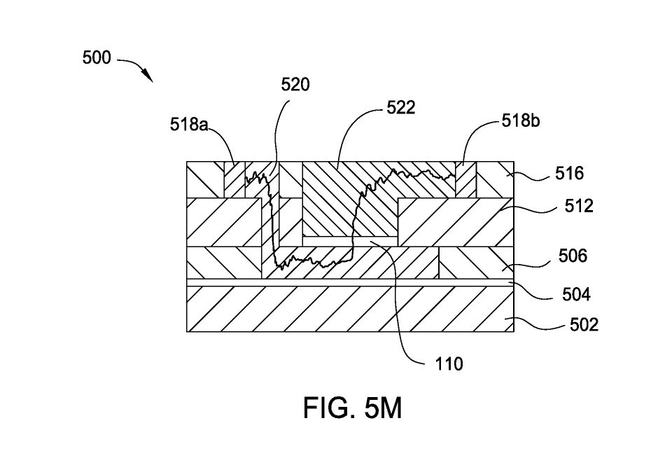

FIGS. 5A-5M depict cross-sectional views of a substrate 500 for biological sequencing applications at various stages of a method disclosed herein. As discussed above, during biological sequencing processes a sample-containing fluid and a sample-free fluid are applied to either side of a nanopore, respectively. FIGS. 5A-5M show a substrate 500 for biological sequencing applications at various stages of a single-sided fabrication process, which provides for both a sample-containing fluid and/or a sample-free fluid to be added from a topside of the substrate.

As shown in FIG. 5A, an oxide layer 504 is deposited over or grown over a first silicon layer 502 of the substrate 500. A first nitride layer 506 is then deposited over the oxide layer 502, as shown in FIG. 5B. An etching process is then used to etch a portion of the nitride layer 506. The etching process is generally any suitable etching process. A second silicon layer 508 is then deposited to fill the previously-etched portion of the first nitride layer 506, as shown in FIG. 5C. Next, a dielectric layer 510 is deposited and patterned over at least a portion of the second silicon layer 508, as shown in FIG. 5D. The dielectric layer is generally any suitable dielectric material, including but not limited to, oxides and nitrides. In one aspect, the dielectric layer 510, such as a metal oxide layer, is atomic layer deposited to a thickness between about 1 nanometer (nm) an about 10 nm, for example, about 5 nm. A second nitride layer 512 is deposited over the remaining first nitride layer 506, the second silicon layer 508, and the dielectric layer 510. An etching process is then used to etch portions of the second nitride layer 512. In the example illustrated in FIG. 5F, a portion of the second nitride layer 512 over the dielectric layer 510 is etched, as well as a portion of the second nitride layer 512 over a portion of the second silicon layer 508. A third silicon layer 514 is then deposited over the etched portions of the second nitride layer 512, as shown in FIG. 5G. Next, as shown in FIG. 5H, a third nitride layer 516 is deposited over the second nitride layer 512 and the third silicon layer 514 of the substrate 500. Portions of the third nitride layer 516 are then etched and filled with a conductive material 518, as shown in FIG. 5I. Portions of the third nitride layer 516 are then etched to expose the second silicon layer 508 and the third silicon layer 514.

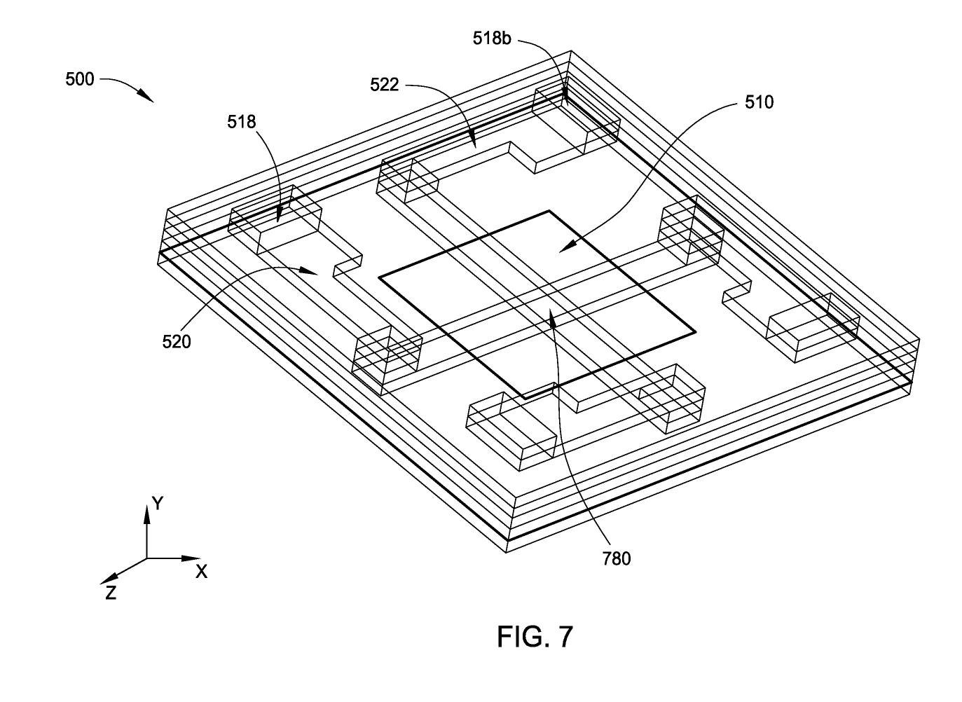

The second silicon layer 508 and the third silicon layer 514 are then selectively etched, as shown in FIG. 5K. For example, radical-based chemistry is used to deliver tunable selectivity for removal of the second silicon layer 508 and the third silicon layer 514 with atomic-level precision. The selected etchant and radicals selectively etch the second silicon layer 508 and the third silicon layer 514. An example of a chamber for performing the selective etching is a Producer® Selectra™ Etch chamber available from Applied Materials, Inc. of Santa Clara, Calif. The selective etch of the second silicon layer 508 and the third silicon layer 514 provides a first bath 520 and a second bath 522, as shown in FIG. 5L. The first bath 520 and the second bath 522 are positioned on either side of the dielectric layer 510, but are able to be filled from the top. For example, the first bath 520 and the second bath 522 are generally filled with a buffer fluid. A voltage is then applied from the first conductive material portion 518a to the second conductive material portion 518b, which provides a dielectric breakdown of a portion of the dielectric layer 510 to form a nanopore 110. If you are filling the two baths 520, 522 with solution, a bubble of air may form near the nanopore 110 trapped by incoming liquid. As shown in FIG. 7, a liquid channel on either side nanopore 110 has an inlet and outlet, so that one or more bubbles are not trapped near the nanopore 110.

The single-sided processes described above allow the first bath 520 and the second bath 522 to be isolated from one another, but also filled from the same side, such as the topside of the substrate 500.

In the examples of FIGS. 5A-5M, various silicon, dielectric, nitride, and conductive layers are described. The method disclosed herein more generally applies to depositing various non-selectively etchable layers and selectively-etchable layers in a stack and selectively etching the selectively-etchable layers to form a first bath and a second bath on the same side of a substrate, the first bath and the second bath being separated by a free-standing membrane, which may have a nanopore therethrough. In further embodiments, wet etch processes are used to form the first bath and the second bath on the same side of the substrate.

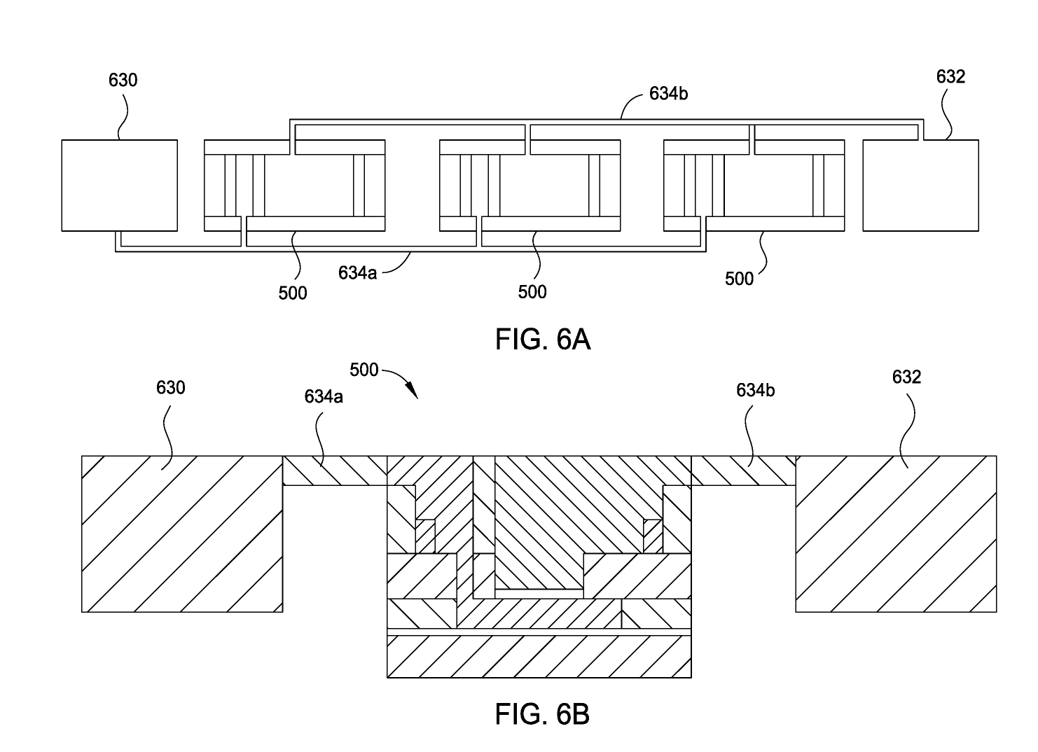

FIG. 6A is a top down view of a plurality of substrates 500 connected to a first bath reservoir 630 and a second bath reservoir 632 by a plurality of channels 634a, 634b. FIG. 6B is a cross-sectional view of one of the substrates 500 connected to the first bath reservoir 630 and the second bath reservoir 632.

In one aspect, the first bath reservoir 630 is generally a reservoir for a sample-containing conductive fluid, such as a DNA-containing conductive fluid reservoir, and the second bath reservoir 632 is generally a reservoir for a sample-free conductive fluid, or vice versa. In another aspect, both the first bath reservoir 630 and the second bath reservoir 632 contain a sample-containing fluid. As shown in FIG. 6A, the plurality of substrates 500 (three are shown) can be fluidically addressed from the common first bath reservoir 630 and the common second bath reservoir 632 through channels 634a and channels 634b, respectively. In one aspect, the channels 634a and 634b are internal channels. Accordingly, multiple baths can be filled by dropping the conductive fluids into larger reservoirs a distance away from the baths. In one aspect, the baths and reservoirs are filled from the topside because of the one sided processing methods described above. The conductive fluid then fills the channels via capillary effect and thus makes its way into the baths of the substrates 500.

While FIGS. 6A-6B show substrates 500 formed according to methods described herein, the methods of fluidically addressing a plurality of nanopore devices from one or more reservoirs is applicable to nanopore devices formed by any suitable manufacturing processes.

Benefits of the present disclosure include the ability to quickly form high volumes of well-controlled nanopores and nanopore arrays, which are generally fluidically addressable from one or both sides of the substrate. Disclosed methods generally provide nanopores that are well-controlled in size through a thin membrane. Methods of manufacturing nanopores of well-controlled size provide improved signal-to-noise ratios because the size of the nanopore is similar to the size of the sample, such as a single strand of DNA, being transmitted through the nanopore, which increases the change in electric current passing through the nanopore.

Methods described herein also provide for vertical or horizontal free-standing membranes for biological applications, such as DNA sequencing, that are thin, for example, less than or equal to 10 nm, dielectric, chemically resistant to saline solutions (KCl), have high selectivity to chemistry of etch processes, are physical and electrical pinhole free, have low stress, and are wettable. The thinner the free-standing membrane, the more the electrical field will concentrate around the edge, thus, the thinness of the free-standing membranes fabricated according to methods described herein allows for high signal-to-noise ratio during use for biological applications, such as DNA base identification.

Even further, the methods and apparatus described herein allow for fluidically addressing different nanopores in different ways. For example, methods described herein provide for simple fluidic addressing of one or more nanopores from a common sample-containing source and a common sample-free source, individually or in combination. Moreover, the formed arrays of nanopore devices can be transported for filling at a location away from the site of manufacture.

While the foregoing is directed to aspects of the present disclosure, other and further aspects of the disclosure may be devised without departing from the basic scope thereof, and the scope thereof is determined by the claims that follow.

What is claimed is:

1. A method for forming a biological sequencing device, comprising:
forming a plurality of nanopore devices on a substrate, each nanopore device having a first bath and a second bath;
forming a first bath reservoir in fluid communication with one or more of the first baths through a plurality of first channels; and
forming a second bath reservoir in fluid communication with one or more of the second baths through a plurality of second channels.

2. The method of claim 1, further comprising:
filling a portion of at least one of the plurality of nanopore devices by filling the first bath reservoir with a sample-containing fluid and flowing the sample-containing fluid through at least one of the plurality of first channels to the at least one of the plurality of nanopore devices.

3. The method of claim 1, wherein the first bath and the second bath of each of the plurality of nanopore devices is on a same side of the substrate.

4. The method of claim 1, further comprising:
filling a portion of the at least one of the plurality of nanopore devices by filling the second bath reservoir with a sample-free fluid and flowing the sample-free fluid through at least one of the plurality of second channels to the at least one of the plurality of nanopore devices.

5. The method of claim 1, wherein each of the plurality of nanopore devices is filled individually.

6. The method of claim 1, wherein two or more of the plurality of nanopore devices are filled collectively.

7. The method of claim 1, wherein each of the plurality of nanopore devices is individually electronically addressable.

8. A method for forming a nanopore device, comprising:
depositing a first selectively-etchable material over a first non-selectively etchable material on a substrate;
depositing a dielectric material over the first selectively-etchable material;
depositing a second selectively-etchable material over the dielectric material;
depositing a second non-selectively etchable material over the second selectively-etchable material; and
selectively etching the first selectively-etchable material and the second selectively-etchable material to form a first bath and a second bath on a single side of the substrate and on either side of the dielectric material.

9. The method of claim 8, wherein selectively etching the first selectively-etchable material and the second selectively-etchable material comprises exposing the substrate to an etchant selected to etch the first selectively-etchable material the second selectively-etchable material over the first non-selectively etchable material and the second non-selectively etchable material.

10. The method of claim 8, further comprising:
filling the first bath and the second bath with a conductive solution.

11. The method of claim 10, further comprising:
applying a voltage from a first portion of conductive material adjacent the first bath to a second portion of conductive material adjacent the second bath to form a nanopore through the dielectric material.

12. The method of claim 11, wherein the nanopore is formed through the dielectric material, the dielectric material being a vertical membrane.

13. The method of claim 8, wherein the first selectively-etchable material and the second selectively-etchable material comprise silicon.

14. The method of claim 8, wherein the first non-selectively etchable material and the second non-selectively etchable material comprise silicon.

15. The method of claim 8, wherein the dielectric material is an oxide or a nitride material.

16. A device for biological sequencing applications, comprising:
a plurality of nanopore devices;
a first bath reservoir; and
a second bath reservoir, the first bath reservoir being fluidically coupled to each of the plurality of nanopore devices through a series of first channels and the second bath reservoir being fluidically coupled to each of the plurality of nanopore devices through a series of second channels.

17. The device of claim 16, wherein each of the plurality of nanopore devices comprises a first bath and a second bath.

18. The device of claim 17, wherein the first bath of each of the nanopore devices is in fluid communication with the first bath reservoir through the series of first channels.

19. The device of claim 17, wherein the second bath of each of the nanopore devices is in fluid communication with the second bath reservoir through the series of second channels.

20. The device of claim 16, wherein each of the plurality of nanopore devices is individually fluidically addressable.

21. The device of claim 16, wherein two or more of the plurality of nanopore devices are addressable in combination.

22. The device of claim 16, wherein each of the plurality of nanopore devices is individually electronically addressable.

23. A method for forming a substrate, comprising:
depositing a first non-selectively etchable material over a second non-selectively etchable material;
etching a portion of the first non-selectively etchable material;
filling the etched portion of the first non-selectively etchable material with a first selectively-etchable material;
depositing a dielectric material over a portion of the first selectively-etchable material;
depositing a third non-selectively etchable material over the first non-selectively etchable material, the first selectively-etchable material, and the dielectric material;
etching a portion of the third non-selectively etchable material;
filling the etched portion of the third non-selectively etchable material with a second selectively-etchable material;
depositing a fourth non-selectively etchable material over the third non-selectively etchable material and the second selectively-etchable material;
etching a portion of the fourth non-selectively etchable material;
filling the etched portion of the fourth non-selectively etchable material with a conductive material; and
selectively etching the first selectively-etchable material and the second selectively-etchable material to form a first bath and a second bath on a topside of the substrate.

24. The method of claim 23, further comprising:
filling the first bath and the second bath with a conductive solution.

25. The method of claim 24, further comprising:
applying a voltage from a first portion of the conductive material to a second portion of the conductive material to form a nanopore through the dielectric material.

26. The method of claim 25, wherein a size of the nanopore is less than 100 nanometers.

27. The method of claim 26, wherein a size of the nanopore is between about 0.5 nanometers and about 5 nanometers.

28. The method of claim 23, wherein the first selectively-etchable material and the second selectively-etchable material comprise silicon.

29. The method of claim 28, wherein selectively etching the first selectively-etchable material and the second selectively-etchable material comprises exposing the substrate to an etchant selected to selectively-etch silicon over the dielectric material.
